(19) United States
(12) Patent Application Publication
(10) Pub. No.: US 2011/0095212 A1
(43) Pub. Date: Apr. 28, 2011

(54) Electrically actuatable clamp

(75) Inventor: Gerhard BRIESKE, Ampfing (DE)

(73) Assignee: LIFEBRIDGE MEDIZINTECHNIK AG, Ampfing (DE)

(21) Appl. No.: 12/910,175

(22) Filed: Oct. 22, 2010

(30) Foreign Application Priority Data
Oct. 23, 2009 (EP) 09013429.7

(51) Int. Cl. A61M 39/28

(52) U.S. Cl. 251/9

(57) ABSTRACT

An electrically actuatable clamp for the clamping of hoses including an electric drive, two scissor levers which can be pivoted relative to one another about a pivot axis between an open position and a clamping position and which have respective clamping surfaces, and a translation device by means of which a movement of the electric drive can be translated into a movement of the scissor levers.

The invention relates to an electrically actuatable clamp for the clamping of hoses.

In medical devices, such as e.g. heart-lung-machines fluids, in particular blood are transported by means of a hose system. A heart-lung machine inter alia serves to take over the function of the heart and the lung of the patient and to maintain the blood circulation, for a certain time frame, e.g. during a heart operation. In this respect it is of large importance that no air bubbles are transported in the systemic circulation of the patient to prevent a life threatening introduction of air into the systemic circulation of the patient. For this reason a bubble detector can be provided at a suitable position in such medical apparatuses. If an air bubble is detected in the extracorporeal blood circuit by the detector, then the circulation must be interrupted immediately so that an endangerment of the patient can be ruled out. For this purpose clamping apparatuses serve which engage at a hose of the fluid transport system and if necessary squeeze these.

In this respect the squeezing of the hose by the clamping apparatus must take place extremely fast and should only have a delay of a few milliseconds up to a 100 milliseconds.

In particular for mobile heart-lung-machines, i.e. portable heart-lung-machines, it is of advantage when such hose clamps can be actuated electrically, for example by means of an electromagnet or an electric motor. In principle this can be caused by a lifting magnet pressing a clamping body directly onto the hose against a stationary holding surface. However, an electromagnet with relatively large dimensions is required to thereby generate the required clamping force and closure speed for a reliable operation which, in particular for portable devices is associated with considerable complications in view of the construction space, the weight and the cost.

It is therefore an object of the invention to provide an electrically actuatable clamp which for a relatively small demand in complexity and cost enables a reliable and sufficiently fast clamping of hoses.

This object is satisfied by a clamp having the features of claim 1. A clamp in accordance with the invention includes an electric drive, two scissor levers, which can be pivoted relative to one another about a pivot axis between an open position and a clamping position and which have respective clamping surfaces, and a translation device by means of which a movement of the electric guide can be translated into a movement of the scissor levers.

The electric drive can e.g. be a simple electromagnet, an electrically actuated linear drive or be an electric motor. The scissor levers having the corresponding clamping surfaces enable a secure and reliable clamping of a hose situated between the clamping surfaces, wherein the clamping force and the closure speed can be simply matched by a corresponding selection of the lever length. Since the electric drive does not act directly on the scissor levers, but rather on the translation device which for its part brings about a movement of the scissor levers, the scissor movement is decoupled from the actual drive movement. Thus, it is in particular possible to bring about a high clamping force using an electric drive having relatively small dimensions and thus to accomplish a reliable clamping of the hose. The clamp in accordance with the invention can, in particular, be adapted as a quick action clamp.

Further embodiments of the invention are described in the dependent claims, the description, as well as in the drawing.

In accordance with one embodiment the translational device translates a rotational movement of the electric drive into a pivot movement of the scissor levers. The translation device can thus be configured such that the rotational movement of a drive shaft is converted into a linear movement of the scissor lever ends. As a result it is possible to drive the clamp by means of a simple and cost-effective electric motor and to thereby do without complex and expensive linear drives.

The translational device can be self-locking in a state associated with the clamping provision of the scissor levers. After a release of the clamp the current supply of the electric drive can be interrupted when such a self-locking state is present without fear that the clamp could be released and the circulation could be started albeit an air bubble being present. Due to the fact that it is not necessary to apply a continuous current to the electric drive for the continuous clamping of the hose, a significant energy saving can be achieved which is advantageous, in particular for mobile apparatuses.

In accordance with a further embodiment the translational device includes a rotational body rotatable about a rotational axis in which cam tracks are formed for the compulsory guidance of end sections of the scissor levers. Such a cam track guide is simple and cost-effective to manufacture and enables a reliable translation of a rotational movement into the desired linear scissor lever movement. The control curve for the opening process or the closure process of the scissor levers is in this respect determined by the run of the cam tracks. In dependence on the embodiment, the cam tracks can be grooves, recesses or slots in the rotational body. The end section of a scissor lever is received in each cam track.

Advantageously the rotational axis is arranged perpendicular to the pivot axis. The rotational body thus rotates in a plane which runs parallel to the pivot axis. In this manner a simple and direct translation of the rotational movement into a linear movement of the scissor lever ends is achieved.

The cam tracks can have an arc-shaped run in a plane running parallel to the pivot axis. In particular, two cam tracks adjacent to one another can be provided which are displaced relative to one another. It is essential that the scissor lever end received in the associated cam track either moves away from the rotational axis or towards the rotational axis depending on its running direction. In dependence on the rotational direction of the rotational body thus a movement directed towards one another or away from another of the scissor levers is achieved and so finally an actuation of the clamp or release of the clamp is achieved.

Furthermore, the cam tracks can have lateral guide surfaces with protruding holding sections for the holding of guide elements in the cam tracks. The guide elements can be cam track followers or similar components which are provided at each of the scissor levers. The lateral guide surfaces can be formed, in particular, such that they partly surround the cam track followers and thereby fix these axially.

In accordance with an embodiment the holding sections vary in size along the cam track. For example, the holding section of an inner lying guide surface becomes smaller with an increase in distance from the rotational axis to thus compensate for the stronger inclination of the scissor levers at this position.

In accordance with a further embodiment the cam tracks have a respective locking section at an end adjacent to the rotational axis which locking sections bring about a retention of the scissor levers in the clamped position. Such a locking section can, for example, be provided in the form of a reinforced curved end region. The reinforced curvature prevents a selfacting back sliding of the scissor levers in the cam tracks. An active rotation of the rotational body into the open direction is in fact required to release a locked clamp. Also actuator bolts or bars can be separately provided in the cam tracks to lock the scissor levers on arriving at the clamping position. Due to the locking function the current supply of the electric drive can be interrupted immediately after arriving at the clamping position to thereby save electrical energy. Due to the mechanic locking of the scissor levers in the clamping position, the application of a holding current is thus not necessary.

The scissor levers can have guide elements which are received in the cam tracks, in particular can have spherically shaped guide elements which are received in the cam tracks. The shape of the guide elements or the cam track followers is matched to the shape of the lateral guide surfaces of the associated cam track. To take into consideration the varying distance between the guide element and the crossing section of the scissor levers during the scissor lever movement, the guide elements can be movably mounted on sliding sections of the scissor levers. For example, a respective end section of a scissor lever can be configured as a cylindrical rod on which the cam track followers slide. For this reason the cam track followers can be provided with cylindrical feed-throughs and be stacked onto the rods.

Preferably the clamping surfaces are aligned parallel to one another in the clamping position. This ensures a uniform clamping force and thus a reliable clamping of the hose. A corresponding alignment of the clamping surface can be achieved in a simple manner by the design of the scissor levers. For the arrangement of the clamping surfaces it can further be taken into account that the hose also has a certain thickness in the clamped state.

In accordance with a further embodiment a drive element of the electric drive acts on an outer diameter of a rotational body of the translational device, in particular of a disc-shaped rotational body of a translational device. For example, the drive shaft of an electric motor can bring about a rotation of the rotational body by means of a corresponding drive disc or a drive gear. For such an embodiment a three-fold force transfer between the drive and the clamping surface takes place. The first force transfer takes place due to the interaction of the drive at the outer circumference of the rotational body. Here a relatively small drive disc can, for example, rotate a relatively large rotational body by means of which a step down in gear is brought about. The second force transfer is achieved by the control cam of the cam shafts whose run influences the force transfer. The third force transfer is finally brought about by the scissor levers themselves, wherein, for example, a relatively high clamping force amplification is possible here due to correspondingly long scissor lever sections at the drive side. As a whole the three-fold force transfer allows the use of a fast, but typically weak electric drive which is advantageous in view of the acquisition costs, construction space and weight.

In accordance with an embodiment of the invention the rotational body has an external toothing with which the drive element of the electric drive, configured as a toothed member, meshes. The outer toothing can be formed directly at the outer circumference of the rotational body. Alternatively, for example a cogwheel can be rotational fixedly connected to the rotational body. For example, a pinion can interact with the outer toothing which pinion is directly attached to the drive shaft. Alternatively, also a gear can be provided between the drive shaft and the pinion. Furthermore, it is also possible to bring about an interaction between a toothed rack and the outer toothing which is then moved by means of a linear drive. A force transfer by means of an outer toothed rotational body works solidly and reliably.

A support frame for the common support of the translational device and a pivot pin of the scissor levers can be provided. This simplifies the mounting of the clamp in the associated medical device. For example, the support frame can be a U-shaped sectional element having a base plate section and two protruding carrier sections, wherein the rotational body is rotationally stored in the base plate section. The pivot pin can then extend through the two carrier sections of the sectional element.

The invention will be described in the following with reference to an embodiment by means of the attached FIGURE.

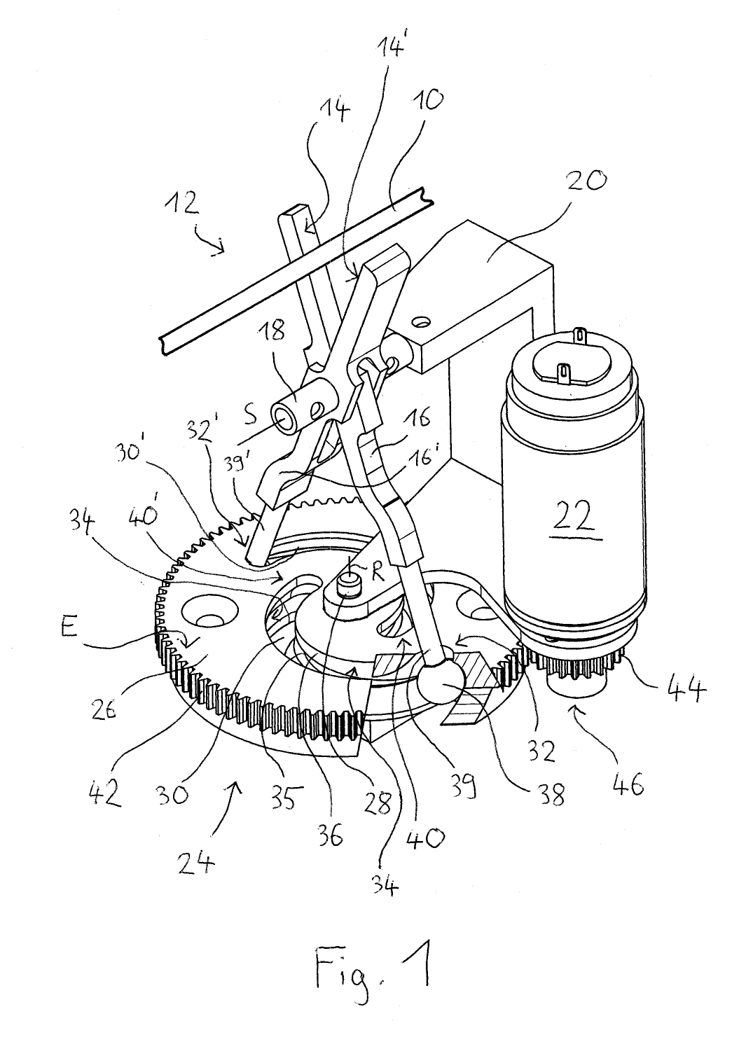

FIG. 1 shows a perspective view of a clamp in accordance with the invention, partially in an open view.

In the perspective view of FIG. 1, 10 refers to a flexible hose which should be clamped under certain circumstances, for example a blood transport hose within a heart-lung-machine. For the on demand clamping of the hose 10, a clamp 12 is provided. As soon as a non-shown detector recognizes that an air bubble is present in the blood flow flowing through the hose 10, the hose clamp 12 must be closed to clamp the hose 10. The clamping is achieved by means of two planar clamping surfaces 14, 14′ which can be formed at respective scissor levers 16, 16′. The scissor levers 16, 16′ are pivotally stored on a pivot pin 18, which itself sits in a fixed support frame 20. The support frame 20 is installed in the associated heart-lung-machine, wherein the hose 10 is guided past the clamping surfaces 14, 14′ of the scissor levers 16, 16′. The pivot pin 18 defines a pivot axis S about which the scissor levers 16, 16′ can be pivoted relative to one another. The position of the two scissor levers 16, 16′ illustrated in the image corresponds to an open position of the hose clamp 12, as the clamping surfaces 14, 14′ are not in contact with the hose 10 here. On movement of the scissor levers 16, 16′ towards one another to press the clamping surfaces 14, 14′ against the hose 10 and to cause an interruption of the blood flow through this. By moving the two scissor levers 16, 16′ towards one another, these are thus transferred into a clamped position.

The scissor levers 16, 16′ can be moved to and fro between the open position and the clamping position by means of an electric motor 22, wherein the translation of the rotational movement of the electric motor 22 into the pivot movement of the scissor levers 16, 16′ is achieved by means of a translational device 24 which is also stored in the support frame 20.

The translational device 24 includes a rotational body 26 which is rotatably stored in the support frame 20 by means of a rotational pin 28. The rotational pin 28 defines a rotational axis R for the rotational body 26 which is arranged perpendicular to the pivot axis S. In the illustrated example, the rotational body 26 has the shape of a disc, in which the two curved slots 30, 30′ are formed. The slots 30, 30′ run within a cam track plane E which is defined by a flat side of the disc-shaped rotational body 26 and extends parallel to the pivot axis S. The scissor levers 16, 16′ are respectively received in one of the slots 30, 30′ at an end section 32, 32′, so that the slots 30, 30′ form cam tracks for the compulsory guidance of the scissor levers 16, 16′. The arc-shaped run of the slots 30, 30′ defines respective control cams for a movement of the scissor lever 16, 16′ towards one another or away from one another.

The end sections 32, 32′ are configured as cylindrical rods 39, 39′ on which spherically shaped cam track followers 38 are slidably movably stored. On rotational movement of the rotational body 26 the cam track followers 38 carry out a linear movement in the cam track plane E of the rotational body 26 in the respective cam track 30, 30′. This linear displacement of the cam track followers 38 for its part causes a pivot movement of the scissor levers 16, 16′ about the fixed pivot axis S. The control cam of the cam tracks 30, 30′ can be selected such that a certain desired connection between the angular position of the rotational body 26 and the degree of opening at the clamp 12 results. For certain applications it is namely desirable when the opening speed of the scissor levers 16, 16′ or the closure speed of the scissor levers 16, 16′ changes in the course of their movement.

The change of separation between the pivot axis S and the cam track followers 38 occurring during the pivot movement of the scissor levers 16, 16′ is compensated by a sliding displacement movement of the cam track followers 38 at the respective rods 39, 39′. On rotation of the rotational body 26, starting from the open position illustrated in FIG. 1, in the counter clock-wise direction the scissor levers 16, 16′ are thus moved towards one another into the clamping position and thus the clamping surfaces 14, 14′ are moved towards one another into the clamping position. The inclination and the end separation of the clamping surface 14, 14′ are selected in this respect, such that in the clamping position a parallel alignment of the clamping surfaces 14, 14′ is present and the separation between the clamping surfaces 14, 14′ corresponds to the residual thickness of the hose 10 which is pressed together.

The slots 30, 30′ have respective lateral guide surfaces 34 in which grooves 35 are formed with correspondingly protruding holding sections 36 for the holding of the cam track followers 38 in the cam tracks 30, 30′. The depth of the grooves 38 and thus the height of the associated holding sections 36 varies along the cam track run, such that the inclination of the rods 39, 39′ in the respective position is compensated. In the position associated with the open position shown in the FIGURE only a relatively weak distinct groove 35 is present.

At an end region of the slots 30, 30′ adjacent to the rotational axis R, the run is more strongly curved in comparison to the residual regions, so that for every slot 30, 30′ a locking section 40, 40′ results in a position associated with the clamping position. The locking sections 40, 40′ ensure a mechanic fixing of the scissor levers 16, 16′ as soon as these arrive in the clamping position. To release and reopen the hose clamp 12 it is thus necessary to cause a moment inertia acting on the rotational body 26 in the clock-wise direction. The translational device is thus self-locking in a state associated with the clamping position of the scissor levers 16, 16′.

The actuation of the rotational body 26 by means of an electric motor 22 will now be described in detail. At the outer circumference of the rotational body 26 an outer toothing 42 is provided which meshes directly with the pinion 44 attached at the motor shaft 46 of the electric motor 22. On applying a current to the electric motor 22 this transfers a moment of inertia onto rotational body 26, wherein a decrease in gear corresponding to the tooth numbers of the outer toothing 42 and also to the pinion 44 is achieved. The gear is matched to the respective application demands. Alternatively, also a toothed rack could be provided instead of the pinion 44 which is moved to and fro by a linear drive or a lifting magnet.

As soon as the previously mentioned detector indicates the presence of air bubbles in the blood flow transported through the hose 10, a corresponding safety signal is generated and due to this the electric motor 22 is provided with electricity. The motor shaft 46 rotationally fixedly turns the pinion 44 connected to it which for its part rotates the rotational body 26 in the counter-clockwise direction about the rotational axis R by means of the outer toothing 42. By means of the slots 30, 30′ and the cam track followers 38 guided in them, finally the pivotal movement of the scissor levers 16, 16′ towards one another is brought about which causes a clamping of the hose 10. Since merely a half turn (90 degrees) of the rotational body 26 is required to move the scissor levers 16, 16′ from the open position into the clamping position a relatively quick clamping can be achieved. The starting moment of inertia of the electric motor 22 can be relatively small, as a high end force can be achieved at the clamping surfaces 14, 14′ by means of the multiple gear ratios. As soon as the clamping position is arrived at, the introduction of current to the electric motor 22 can be stopped or minimized, as the scissor levers 16, 16′ are retained by means of the locking sections 40, 40′ and an opening of the scissor levers 16, 16′ is thus not to be feared. In this manner substantial amounts of electrical energy can be saved which is particularly advantageous for portable heart-lung-machines—which are also powered by battery.

### LIST OF REFERENCE NUMERALS

10 hose
12 hose clamp
14, 14′ clamping surface
16, 16′ scissor lever
18 pivot pin
20 support frame
22 electric motor
24 translation device
26 rotational body
28 rotational pin
30, 30′ slot
32, 32′ end section
34 lateral guide surface
35 groove
36 holding section
38 cam track follower
39, 39′ rod
40, 40′ locking section
42 outer toothing
44 pinion
46 drive shaft
S pivot axis
R rotational axis
E cam track plane

1. An electrically actuatable clamp (12) for the clamping of hoses (10), which includes:
an electric drive (22),
two scissor levers (16, 16′), which can be pivoted relative to one another about a pivot axis (S) between an open position and a clamping position and which have respective clamping surfaces (14, 14′), and
a translation device (24) by means of which a movement of the electric drive can be translated into a movement of the scissor levers (16, 16′).

2. A clamp in accordance with claim 1 wherein the translation device (24) translates a rotational movement of the electric drive (22) into a pivot movement of the scissor levers (16, 16′).

3. A clamp in accordance with claim 1 wherein the translation device (24) is self-locking in a state associated with the clamping position of the scissor levers (16, 16′).

4. A clamp in accordance with claim 1 wherein the translation device (24) includes a rotational body (26) rotatable about a rotational axis (R) in which cam tracks (30, 30′) are formed for the compulsory guidance of end sections (32, 32′) of the scissor levers (16, 16′).

5. A clamp in accordance with claim 4 wherein the rotational axis (R) is arranged perpendicular to the pivot axis (S).

6. A clamp in accordance with claim 4 wherein the cam tracks (30, 30′) have an arc shaped run in a plane (E) extending parallel to the pivot axis (S).

7. A clamp in accordance with claim 4 wherein the cam tracks (30, 30′) have lateral guide surfaces (34) with protruding holding sections (36) for the holding of guide elements (38) in the cam tracks (30, 30′).

8. A clamp in accordance with claim 7 wherein the holding sections (36) vary in size along the cam track run.

9. A clamp in accordance with claim 4 wherein the cam tracks (30, 30′) have a respective locking section (40, 40′) at an end adjacent the rotational axis (R) which locking section brings about a retention of the scissor levers (16, 16′) in the clamped position.

10. A clamp in accordance with claim 4 wherein the scissor levers (16, 16′) have guide elements, in particular spherically shaped guide elements (38) which are received in the cam tracks (30, 30′).

11. A clamp in accordance with claim 1 wherein guide elements (38) are moveably mounted on sliding sections (39, 39′) of the scissor levers (16, 16′).

12. A clamp in accordance with claim 1 wherein the clamping surfaces (14, 14′) are aligned parallel to one another in the clamping position.

13. A clamp in accordance with claim 1 wherein a drive element (44) of the electric drive (22) acts on an outer diameter of a rotational body (26) of the translation device (24), in particular of a disc shaped rotational body (26) of the translation device (24).

14. A clamp in accordance with claim 13 wherein the rotational body (13) has external toothing (42) with which the drive element (22) of the electric drive (22) configured as a toothed member meshes.

15. A clamp in accordance with claim 1 wherein a support frame (20) for the common support of the translation device (24) and a pivot pin (18) of the scissor levers (16, 16′) is provided.
